(19) United States
(12) Patent Application Publication
(10) Pub. No.: US 2016/0114134 A1
(43) Pub. Date: Apr. 28, 2016

(54) Medical Adhesive Tape for Infusion Tubing

(71) Applicant: Dongguan Yitong Weaving Co., LTD., Dongguan (CN)

(72) Inventor: Zuncai YI, Dongguan (CN)

(73) Assignee: Dongguan Yitong Weaving Co., LTD.

(21) Appl. No.: 14/520,864

(22) Filed: Oct. 22, 2014

(51) Int. Cl. A61M 25/02

(52) U.S. Cl. CPC A61M 25/02; A61M 2025/0266

(57) ABSTRACT

A medical adhesive tape comprises a film substrate having a top surface including a loop member surface and a bottom surface including a first adhesive layer provided which is adhered to a release liner, a strip-shaped tearing member provided on a middle section of the loop member surface of the top surface of the film substrate which has a bottom surface defining a hook member surface and defines two ends at which two positioning holes are provided respectively, and two positioning pads secured to the two positioning holes respectively, wherein the loop member surface and the hook member surface are arranged to adhere together. When used, an infusion tubing penetrates through a cavity between the film substrate and the strip-shaped tearing member at one or both ends and is positioned on top of the positioning pad for securing the infusion tubing into position, which is convenience for the user.

### BACKROUND OF THE PRESENT INVENTION

1. Field of Invention

The present invention relates to an accessory item for medical device, and more particularly to an adhesive tape for medical use.

2. Description of Related Arts

Conventional medical adhesive tape for infusion tubing comprises one adhesive layer which is adhered to a releasing liner. When used, the health care worker removes the releasing liner from the adhesive layer of the medical adhesive tape, then the medical adhesive tape is used to secure the infusion pillow which is connected to the blood vessel of a patient or to secure the infusion tubing which is connected to the infusion pillow. If the user use his or her hand to remove the medical adhesive tape by removing the adhesive layer from the releasing liner, then the side of the adhesive layer will be contaminated. If the user wears protective gloves to remove the medical adhesive tape by removing the adhesive layer from the releasing liner, then the gloves will adhere to the adhesive layer. Accordingly, great inconvenience and waste of medical supplies such as gloves and adhesive tape are resulted. This also creates the problem of failure to provide two-way protection.

US Pat. No. 7,793,892B1 discloses a positioning strip for connecting tube which comprises a fixed end and an open end. When used, the open end is tear off and the connecting tube is penetrated through the positioning strip and secured onto the positioning strip. When one connecting tube is secured through the positioning strip and a second connecting tube is required, the open end is required to tear off and then the first connecting tube is loosened or is freely to slide. As a result, the needle at the other end of the connecting tube will tilt or fall off, which is dangerous to the user. In addition, the second connecting tube has to place across the first connecting tube which forms a crossing pattern in order to fixing the connecting tube securely into position, which is very inconvenience to the user. The problem is particularly prominent during operation because a few seconds may be very important to the patient and affect the well-being of the patient. Therefore, there is a need to provide an improvement to solve the above problem.

Accordingly, there is an urgent and immediate need to provide a medical adhesive tape to solve the problems in the existing technologies.

### SUMMARY OF THE PRESENT INVENTION

An object of the present invention is to provide a medical adhesive tape which includes a film substrate, a loop member surface on the film substrate and a strip-shaped tearing member affixed on the middle section of the film substrate in such a manner that the two ends of the strip-shaped tearing member defines an open-state. When used, only one end of the strip-shaped tearing member 4 is required and tear off to secure one infusion tubing while both two ends of the strip-shaped tearing member 4 are tear off to secure two infusion tubing through which the infusion tubing penetrates through the film substrate. The two ends, which is arranged for securing two different infusion tubing, will not cause interference and will not affect the use of each other.

In order to achieve the above object, the followings are employed:

A medical adhesive tape for infusion tubing comprising a film substrate having a top surface and a bottom surface, and a first adhesive layer on the bottom surface of the film substrate, characterized in that: the medical adhesive tape comprises:

a release liner adhered to the first adhesive layer;

a loop member surface is provided on the top surface of the film substrate;

a strip-shaped tearing member affixed onto the middle section of the loop member surface, which comprises: a hook member surface on a bottom surface of the strip-shaped tearing member; two positioning holes in two ends of the strip-shaped tearing member respectively; and two positioning pads secured onto the strip-shaped tearing member through the two positioning holes respectively, wherein the loop member surface and the hook member surface are adhered together defining a fabric hook and loop fastener.

Preferably, the two positioning pads are received inside the two positioning holes respectively.

Preferably, each of the positioning pads is securely positioned at one end of the corresponding positioning hole at a position proximal to the middle section of the strip-shaped tearing member.

Preferably, each of the positioning pads is securely positioned at one end of the corresponding positioning hole at a position farthest from the middle section of the strip-shaped tearing member.

Preferably, each of the positioning pads is securely positioned between two ends of the corresponding positioning hole in a discrete manner.

Preferably, each of the positioning pads is constructed into a rectangular-shaped structure, a round-shaped structure, a semi-circular-shaped structure, a triangular shaped structure, a quadrilateral-shaped structure or a polygonal-shaped structure.

Preferably, each of the positioning pads has a bottom surface which comprises a hook member surface on the bottom surface of the positioning pad.

Preferably, each of the positioning holes has a rectangular-shaped construction, a round-shaped construction, a semi-circular-shaped construction, a triangular shaped construction, a quadrilateral-shaped construction or a polygonal-shaped construction.

Preferably, the strip-shaped tearing member is sewed onto the middle section of the loop member surface.

Preferably, the strip-shaped tearing member is tied onto the middle section of the loop member surface.

Preferably, the strip-shaped tearing member is bonded onto the middle section of the loop member surface.

Preferably, the two ends of the strip-shaped tearing member further comprises two extended portions respectively.

Preferably, each of the extended portion comprises a second adhesive layer coated on a bottom surface of the extended portion.

Preferably, a second releasing liner is provided to adhere to the second adhesive layer for each the second adhesive layer.

Preferably, the first releasing liner is outward extended from the film substrate and exposed from the film substrate.

Preferably, an adhesive is coated onto two ends of the top surface of the film substrate and a third releasing liner is provided to adhere to the two ends of the top surface of the film substrate.

According to the preferred embodiment of the present invention, a medical adhesive tape comprises a film substrate having a top surface and a bottom surface, a first adhesive layer provided on the bottom surface of the film substrate , a release liner adhered onto the first adhesive layer, a loop member surface of a fabric hook and loop fastener on the top surface of the film substrate, a strip-shaped tearing member provided on a middle section of the loop member surface of the top surface of the film substrate, wherein the strip-shaped tearing member has a bottom surface defining a hook member surface of the fabric hook and loop fastener and defines two ends at which two positioning holes are provided at the two ends respectively, wherein two positioning pads are secured to the two positioning holes respectively, and the loop member surface and the hook member surface are adhered together. Compared to conventional arts, the medical adhesive tape according to the preferred embodiment of the present invention has the following advantageous effect: When used, the release liner on the first adhesive layer are removed and the first adhesive layer is adhered onto a skin, an infusion tubing is then arranged to penetrate through a cavity defined between the film substrate and the strip-shaped tearing member, which is positioned on top of the positioning pad. The positioning pad can be flexibly provided on the loop member surface of the film substrate based on a size of the infusion tubing and provide a positioning effect for the infusion tubing. Therefore, the infusion tubing is prevented from sliding movement towards the two ends of the film substrate and an enhanced stability is provided. Then, the hook member surface of the strip-shaped tearing member and the loop member surface are adhered together. Since the strip-shaped tearing member is secured into position through its middle section and its two ends are in open-state, only one end of the strip-shaped tearing member is required to tear off to secure one infusion tubing while both two ends of the strip-shaped tearing member are tear off to secure two infusion tubing, which is very convenience to use. The two ends, which is arranged for securing two different infusion tubing, will not cause interference and will not affect the use of each other. In addition, the structural construction is simple and is suitable for large scale production and use.

### BRIEF DESCRIPTION OF THE DRAWINGS

The present invention is further illustrated in the following figures, which are exemplary only and not intended to be limiting.

FIG. 1 is an illustration of a medical adhesive tape for infusion according to the preferred embodiment of the present invention.

FIG. 2 is an illustration of a medical adhesive tape for infusion according to another preferred embodiment of the present invention.

FIG. 3 is an illustration of a medical adhesive tape for infusion according to another preferred embodiment of the present invention.

### DETAILED DESCRIPTION OF THE PREFERRED EMBODIMENT

The present invention is further described in details with the accompanying figures and embodiments.

### Embodiment 1

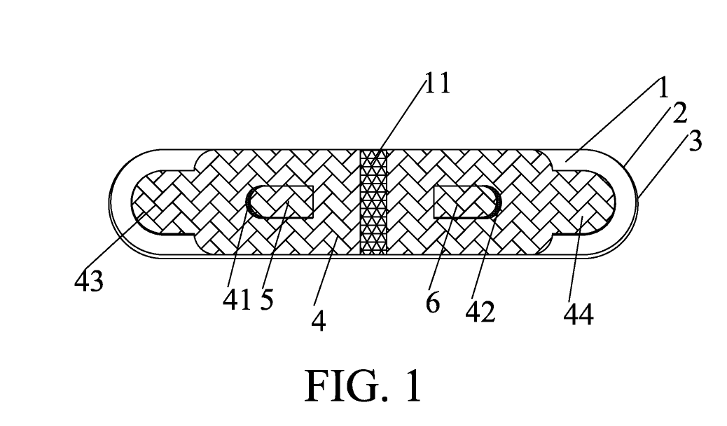

Referring to FIG. 1 of the drawings, a medical adhesive tape according to this preferred embodiment of the present invention comprises a film substrate 1 having a top surface and a bottom surface, a first adhesive layer 2 provided on the bottom surface of the film substrate 1, a release liner 3 (preferably a release paper 3) adhered onto the first adhesive layer 2, a loop member surface of a fabric hook and loop fastener on the top surface of the film substrate 1, a strip-shaped tearing member 4 mounted on a middle section of the loop member surface 11 of the top surface of the film substrate 1, the strip-shaped tearing member 4 has a bottom surface defining a hook member surface of the fabric hook and loop fastener and defines two ends at which two positioning holes 41, 42 are provided at the two ends respectively, and the loop member and the hook member are adhered together. Two positioning pads 5, 6 are received inside the two positioning holes 41, 42 respectively. Each of the two positioning pads 5, 6 are securely connected onto the corresponding positioning holes 41 (42) through one end proximal to the middle section of the strip-shaped tearing member 4. When used, the release liner 3 on the first adhesive layer 2 are removed and the first adhesive layer 2 is adhered onto a skin, an infusion tubing is then arranged to penetrate through a cavity defined between the film substrate 1 and the strip-shaped tearing member 4, which is positioned on top of the positioning pad 5 or the positioning pad 6. The positioning pad 5 or the positioning pad 6 can be flexibly provided on the loop member surface of the film substrate 1 based on a size of the infusion tubing and provide a positioning effect for the infusion tubing. Then, the hook member surface 41 of the strip-shaped tearing member 4 and the loop member surface 11 are adhered together such that the infusion tubing is secured into position. The positioning pads 5, 6 are positioned below the infusion tubing for providing a buffer and protection effect, which can increase the comfort while providing positioning function.

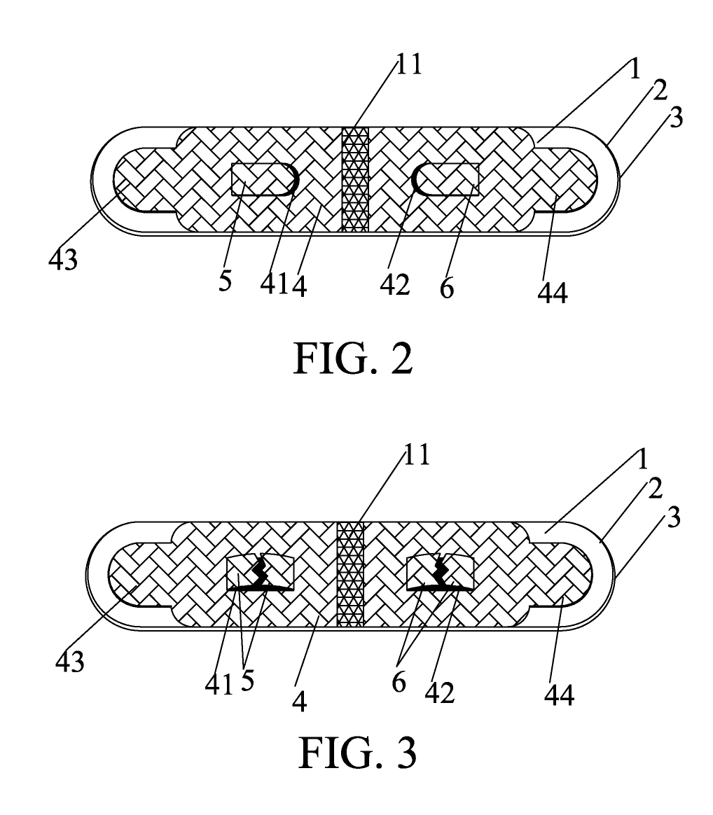

Referring to FIG. 2 of the drawings, each of the two positioning pads 5, 6 are securely connected onto the corresponding positioning holes 41 (42) through one end distal from the middle section of the strip-shaped tearing member 4. The positioning pads 5, 6 are positioned below the infusion tubing for providing a buffer and protection effect and can increase the comfort. On the other hand, the positioning pads 5, 6 can also provide positioning function to prevent sliding movement of the infusion tubing towards the two sides of the film substrate 1 such that an enhanced positioning effect is achieved.

Referring to FIG. 3 of the drawings, each of the two positioning pads 5, 6 are securely connected onto one end of the corresponding positioning holes 41 (42) respectively in a discrete manner. The positioning pads 5, 6 are positioned below the infusion tubing for providing a buffer and protection effect and can increase the comfort. On the other hand, the positioning pads 5, 6 can also provide positioning function to prevent sliding movement of the infusion tubing towards the two sides of the film substrate 1 such that an enhanced positioning effect is achieved.

Preferably, the bottom surface of the positioning pads 5, 6 is a hook member surface such that the positioning pads 5, 6 can be easily adhered onto the loop member surface 11.

Preferably, the positioning holes 41, 42 are constructed into a rectangular-shaped structure, a round-shaped structure, a semi-circular-shaped structure, a triangular shaped structure or a quadrilateral-shaped structure for fitting to an infusion tubing of a particular size in such a manner that a small pressure is applied on the infusion tubing while at the same time the positioning holes 41, 42 can provide a superior positioning effect on the infusion tubing. In addition, a low physical stimulation effect on a skin surface is resulted. Preferably, the rectangular-shaped structure, the semi-circular-shaped structure or their combination is selected.

Preferably, the positioning pads 5, 6 are constructed into a rectangular-shaped structure, a round-shaped structure, a semi-circular-shaped structure, a triangular shaped structure, a quadrilateral-shaped structure or a polygonal-shaped structure.

Preferably, the two ends of the strip-shaped tearing member further comprises two extended portions 43, 44 respectively. Each of the two extended portions 43, 44 has a bottom surface on which a second adhesive layer is coated while a second releasing liner member is provided and adhered onto the second adhesive layer. When used, the second releasing liner is removed to expose the bottom surface of the two extended portions 43, 44, which is an adhesive surface. The infusion tubing is then arranged to penetrate through a cavity defined between the film substrate 1 and the strip-shaped tearing member 4, which is positioned on top of the positioning pad 5 or the positioning pad 6. Then, the two extended portions 43, 44 are adhered to the two ends of the top surface of the film substrate 1 such that the infusion tubing is secured into position.

Preferably, the release liner 3 is outwardly extended from the two ends of the film substrate 1 such that the lease liner 3 can be removed easily when used, thereby the convenience for use is increased.

Preferably, the strip-shaped tearing member 4 is sewed to the middle section of the loop member surface 11.

Preferably, the strip-shaped tearing member 4 is tied onto the middle section of the loop member surface 11 to secure into position.

Preferably, the strip-shaped tearing member 4 is bonded onto the middle section of the loop member surface 11 to secure into position.

Preferably, the two ends of the top surface of the film substrate 1 comprises an adhesive layer and a releasing liner member adhered onto the adhesive layer. When the releasing liner member is removed, the adhesive layer can adhere to the two extended portions 43, 44 of the strip-shaped tearing member 4, which has an adhesive surface layer, to secure the infusion tubing into position.

Compared to conventional arts, the medical adhesive tape according to the preferred embodiment of the present invention has the following advantageous effect: When used, the release liner 3 on the first adhesive layer 2 are removed and the first adhesive layer 2 is adhered onto a skin, an infusion tubing is then arranged to penetrate through a cavity defined between the film substrate 1 and the strip-shaped tearing member 4, which is positioned on top of the positioning pad 5 or the positioning pad 6. The positioning pad 5 or the positioning pad 6 can be flexibly provided on the loop member surface of the film substrate 1 based on a size of the infusion tubing and provide a positioning effect for the infusion tubing. Therefore, the infusion tubing is prevented from sliding movement towards the two ends of the film substrate 1 and an enhanced stability is provided. Then, the hook member surface 41 of the strip-shaped tearing member 4 and the loop member surface 11 are adhered together. When used, since the strip-shaped tearing member 4 is secured into position through its middle section and its two ends are in open-state, only one end of the strip-shaped tearing member 4 is required and tear off to secure one infusion tubing while both two ends of the strip-shaped tearing member 4 are tear off to secure two infusion tubing, which is very convenience to use. The two ends, which is arranged for securing two different infusion tubing, will not cause interference and will not affect the use of each other. In addition, the structural construction is simple and is suitable for large scale production and use.

One skilled in the art will understand that the embodiment of the present invention as shown in the drawings and described above is exemplary only and not intended to be limiting.

It will thus be seen that the objects of the present invention have been fully and effectively accomplished. It embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A medical adhesive tape for infusion tubing comprising a film substrate having a top surface and a bottom surface, and a first adhesive layer on the bottom surface of the film substrate, characterized in that: said medical adhesive tape comprises:
a release liner adhered to the first adhesive layer;
a loop member surface is provided on the top surface of the film substrate;
a strip-shaped tearing member affixed onto the middle section of the loop member surface, which comprises: a hook member surface on a bottom surface of said strip-shaped tearing member; two positioning holes in two ends of said strip-shaped tearing member respectively; and two positioning pads secured onto said strip-shaped tearing member through said two positioning holes respectively,
wherein said loop member surface and said hook member surface are adhered together defining a fabric hook and loop fastener.

2. The medical adhesive tape for infusion tubing according to claim 1, wherein said two positioning pads are received inside said two positioning holes respectively.

3. The medical adhesive tape for infusion tubing according to claim 2, wherein each said positioning pad is securely positioned at one end of said corresponding positioning hole at a position proximal to the middle section of said strip-shaped tearing member.

4. The medical adhesive tape for infusion tubing according to claim 2, wherein each said positioning pad is securely positioned at one end of said corresponding positioning hole at a position farthest from the middle section of said strip-shaped tearing member.

5. The medical adhesive tape for infusion tubing according to claim 2, wherein each said positioning pad is securely positioned intermittently between two ends of said corresponding positioning hole.

6. The medical adhesive tape for infusion tubing according to claim 1, wherein each of said positioning pads is constructed into a rectangular-shaped structure, a round-shaped structure, a semi-circular-shaped structure, a triangular shaped structure, a quadrilateral-shaped structure or a polygonal-shaped structure.

7. The medical adhesive tape for infusion tubing according to claim 1, wherein each of said positioning pads has a bottom surface which comprises a hook member surface on said bottom surface of said positioning pad.

8. The medical adhesive tape for infusion tubing according to claim 1, wherein each of said positioning holes has a rectangular-shaped construction, a round-shaped construction, a semi-circular-shaped construction, a triangular shaped construction, a quadrilateral-shaped construction or a polygonal-shaped construction.

9. The medical adhesive tape for infusion tubing according to claim 1, wherein said strip-shaped tearing member is sewed onto said middle section of said loop member surface.

10. The medical adhesive tape for infusion tubing according to claim 1, wherein said strip-shaped tearing member is tied onto said middle section of said loop member surface.

11. The medical adhesive tape for infusion tubing according to claim 1, wherein said strip-shaped tearing member is bonded onto said middle section of said loop member surface.

12. The medical adhesive tape for infusion tubing according to one of claim 1, wherein said two ends of said strip-shaped tearing member further comprises two extended portions respectively.

13. The medical adhesive tape for infusion tubing according to claim 12, wherein each said extended portion comprises a second adhesive layer coated on a bottom surface of said extended portion.

14. The medical adhesive tape for infusion tubing according to claim 13, further comprising a second releasing liner adhered to said second adhesive layer for each said second adhesive layer.

15. The medical adhesive tape for infusion tubing according to claim 14, wherein said first releasing liner is outward extended from said film substrate and exposed from said film substrate.

16. The medical adhesive tape for infusion tubing according to claim 1, wherein an adhesive is coated onto two ends of said top surface of said film substrate and a third releasing liner is provided to adhere to said two ends of said top surface of said film substrate.
